(19) United States
(12) Patent Application Publication
(10) Pub. No.: US 2005/0049581 A1
(43) Pub. Date: Mar. 3, 2005

(54) Hybrid organ circulatory system

(76) Inventor: Joerg C. Gerlach, Pittsburgh, PA (US)

Correspondence Address: Joerg C. Gerlach MD, PhD, 3613 Butler St., Pittsburgh, PA, 15201, US

(21) Appl. No.: 10/866,127

(22) Filed: Jun. 12, 2004

(30) Foreign Application Priority Data
Jun. 13, 2003 (DE) 103 26 749.2

(51) Int. Cl. A61B 17/00

(52) U.S. Cl. 606/1

(57) ABSTRACT

This invention refers to a hybrid circulatory system with which the transportation of cells and/or substances within a biological organism can be mimicked, in particularly the human body. The natural example is blood with plasma, transferring substances and blood cells, e.g. from hematopoietic-, immune-, and stem cell systems. Such circulatory systems are essential in the development of methods in cell biology, medical therapy, regenerative medicine, tissue engineering, and stem cell applications. Such systems can provide cells for extracorporeal organ-systems, e.g. bio-artificial liver support. Likewise, cells can be prepared and produced, especially progenitor cells for cell transplantation in cell-based therapy. These systems are generally of interest for the production of certain types of cells or metabolic products like mediators, effectors, antibodies, proteins, vaccines and such; whereby organ typical cells can be cultivated, differentiated, and propagated, while communication between cells of different location plays a role, e.g. hybrid bone marrow.

This invention refers to a hybrid circulatory system with which the transportation of cells and/or substances within a biological organism can be mimicked, in particularly the human body. The natural example is blood with plasma, transferring substances and blood cells, e.g. from hematopoietic-, immune-, and stem cell systems. Such circulatory systems are essential in the development of methods in cell biology, medical therapy, regenerative medicine, tissue engineering, and stem cell applications. Such systems can provide cells for extracorporeal organ-systems, e.g. bio-artificial liver support. Likewise, cells can be prepared and produced, especially progenitor cells for cell transplantation in cell-based therapy. These systems are generally of interest for the production of certain types of cells or metabolic products like mediators, effectors, antibodies, proteins, vaccines and such; whereby organ typical cells can be cultivated, differentiated, and propagated, while communication between cells of different location plays a role, e.g. hybrid bone marrow.

Devices for metabolic exchange, e.g. bioreactors, cell perfusion devices, and general modules, especially for liver support systems, are already known as alternative method for animal experiments, the production of biological cell products, or in the area of organ support.

A particularly effective module is described in the EP 059 034 A2 (Gerlach, J. C.)/U.S. Pat. No. 08/117,429: 1993. The described module for the culture and utilization of metabolic performance and/or maintenance of microorganisms consists of a casing with at least three independent membrane systems arranged inside. Of these membrane systems at least two independent membrane systems are developed as hollow fiber membranes. These hollow fiber membranes form a tightly packed 3D interwoven spatial network. The microorganisms are immobilized in the cell compartment of the network and/or to the hollow fiber membrane surfaces.

A first independent hollow fiber membrane system serves for the media inflow. A second independent hollow fiber membrane system serves for the gas supply of the microorganisms with oxygen, and the removal of CO². The outflow of the media is guaranteed through a third independent membrane system.

Each individual, independent hollow fiber membrane system consists of a multitude of individual hollow fiber membranes, whereby the hollow fibers of a system communicate through at least one inflow head, respectively one inflow and outflow head. Thereby, the simultaneous media supply through the inflow head to the hollow fibers of each independent system is guaranteed. Furthermore, the individual hollow fibers are interwoven with each other.

These independent hollow fiber membrane systems create a multi-compartment system in a spatial, tightly packed, interwoven network inside the module in such a way that almost anywhere in the network the organisms have almost identical conditions for substrate supply. Therewith, the conditions in physiological organs with arteries and veins, e.g. the liver, with the arrangement of hepatocytes in lobules are largely simulated. Through the independent arrangement of different membrane systems the module presents the advantage of a decentralized transport of nutrients, products for synthesis, gases, to/from a multitude of microorganisms independent of their position inside the module, the same way as it is in the cell environment of natural organs. The outflow of media is ensured through the third independent membrane system. This membrane system can be a hollow fiber membrane, an exchangeable flat membrane, or an exchangeable capillary membrane. It is crucial that the third membrane system is independent form the other two hollow fiber membrane systems.

One design suggests that the tightly packed network in the inside is constructed from independent hollow fiber membrane systems. In this case all independent membrane systems are hollow fiber membrane systems that are arranged in the inside. Here, one independent hollow fiber membrane system serves the inflow of media, a second hollow fiber membrane system serves the outflow of media, and a third system serves for the supply of other substances, e.g. oxygen. The tightly packed network consists of these three independent systems. Alternative to mass exchange from one to the other hollow fiber membrane system is their use for counter-directional flow operation.

The tightly packed network can be constructed in various ways as long as it is guaranteed that the microorganisms inside receive an identical substrate supply. The tightly packed network can consist of, for instance, tightly packed layers each with alternating layers of independent systems. The second layer, also consisting of individual hollow fiber membranes, is arranged on the same plane, however opposite the first layer, rotated by, for example, 90 degrees.

These layers alternate and create a dense package. The third independent hollow fiber membrane system that, once again, consists of individual layers of hollow fiber membranes, infuses the first two layers vertically form top to bottom and thereby “interweaves” the first two independent layers.

A further design plans for three independent hollow fiber membrane systems with alternating, overlaying layers that are all arranged in one plane but each rotated about 60 degrees.

This tightly packed network is arranged inside the module. Because each independent system communicates with at least one inflow, respectively one inflow and outflow, even distribution of inflowing media as well as steady oxygenation is ensured. Through the third independent system for the outflow of media, the media can continuously and consistently be eliminated from anywhere in the module.

In a further design, in addition to the three hollow fiber membrane systems, an additional independent membrane system is used inside the module for media outflow. For that purpose and exchangeable flat membrane or an exchangeable capillary membrane can be mounted on the outer casing.

A further design plans that the tightly packed network is generated from two independent hollow fiber membrane systems, whereby one serves for the inflow of media and the other for oxygenation. A third independent membrane system, which is an exchangeable flat-or capillary membrane and mounted on the outer casing serves for the outflow of media.

The tightly packed network in the inside, which is generated from the two hollow fiber membrane systems, is designed analogous afore described systems.

The use of hydrophilic or hydrophobic polypropylene, polyamid, polysuphon, cellulose, or silicon-rubber is preferred for hollow fiber membranes. The selection of hollow fiber membranes depends on the molecules planned for substance exchange. However, all state of the art hollow fiber membranes, known as substance exchange devices (or mass exchange devices), can be used.

By using three independent hollow fiber membrane systems, which form a tightly packed network, a capillary system of fluid impermeable capillaries, e.g. stainless steel or glass can be used, which can serve to control the temperature inside the module. This system also facilitates the even cooling of the module, its inside and the infuse microorganisms, below −20 degrees Celsius. In a further design all other hollow fiber membrane systems can also be used for temperature control, respectively cool down below the freezing point.

In a further design the outer casing is made from a poured cast, whereby it is ensured that an access way form outside into the volume of the capillaries is possible.

In another design the module exhibits various additional access ways. One access way serves as inflow device for microorganisms into the module. Additional access ways serve for instance for pressure-, pH-, and temperature measurements inside the modules.

This bioreactor already exhibits excellent results in regards to substrate supply and substrate removal. A further module that has been submitted simultaneously on the same day, by the same inventors, along with this registration is known as “Module for the culture and utilization of metabolic performance and/or for the maintenance of microorganisms”(German patent application #103 26 744.1 of 13 Jun. 2003, J. Gerlach). This module consists of a body that is arranged inside a water-/germ tight container, whereby the body is designed with open pores that can communicate with each other. Simultaneously this body exhibits at least one channel like hollow pathway system whose individual hollow pathways infuse the body and intersect and/or overlay each other. Because the body inside the container is made of porous material, whose pores can communicate with each other, the connection between the pores via their connections to the independent, channel like hollow pathway systems is guaranteed. Inside the module, microorganisms, particularly cells, inside the pores of this porous body, are immobilized without completely filling it up. Through the independent, channel like, hollow pathway systems, arranged inside the body, a consistent supply and waste disposal of the microorganisms inside the open pores, especially the cells, can occur from anywhere in the body with a low substrate gradient. Because the arrangement of the pathways, mass exchange is comparable to the module described above. The hollow fiber membranes and the hollow channel-like pathways with their walls to the open porous body serve comparable functions. This module replicates the organ supply similar to the natural organ. With this module, because of the open pores of the hollow pathway system, a bioreactor is available that facilitates an optimal substrate supply and removal of a relatively large amount of microorganisms over longer periods of time.

A channel like hollow pathway system is advantageously developed in such a way that it consists of collateral channels arranged in one plane. It is advantageous if the channel like hollow pathway system consists of several such planes that overlay each other in a predetermined distance. The distance between the individual channels of a hollow pathway system in a plane and between the individual planes is preferably in the range from 0.5-5 mm. The diameter of the individual channels is preferably 0.1-3 mm. The body of the module can exhibit at least two such hollow pathway systems that intersect and/or overlay each other.

This facilitates a substrate exchange across both hollow pathway systems, respectively between both hollow pathway systems, via counter current flow and therefore with relatively high mass exchange capacity and low substrate gradients.

An advantageous design is arranged with intersecting hollow pathway systems. Therefore one hollow pathway system, preferably consisting of several overlaying planes, infuses the body form on direction, and the second hollow pathway system infuses the body in the other direction at, for example, a 90 degree angle. Because the planes are arranged in afore mentioned distance the supply and removal of substrate from the microorganisms, inside the pores of the open porous body, is guaranteed almost anywhere in the body. This module naturally includes all additional designs in regards to the geometrical arrangement of the hollow pathway systems to each other, provided that an almost identical substrate supply and removal process from anywhere is the is secured. The two hollow pathway systems can intersect inside the body at a predetermined angle. They can also be arranged parallel on top of each other whereby the counter current principle is optimally utilized.

If the module exhibits a third independent hollow pathway system, it is preferably constructed from parallel-arranged hollow pathways in another plane. These hollow pathway systems also infuse the body, for instance, vertically form top to bottom, interweaving the first two independent hollow pathway systems with each other and integrating other decentralized functions like oxygenation or CO₂ removal.

The module with the third hollow pathway system includes all geometrical arrangements, provided that an almost identical substrate supply and removal process for the microorganisms, meaning the cells, is secured from anywhere in the body. Analogous a fourth or additional hollow pathway system can be integrated, whereby additional functions like cell drainage, cell injection, cell extraction, and movement/pressure/flow application for cell removal are made possible.

In this module the first independent hollow pathway system can serve for media inflow. The second independent hollow pathway system serves for the supply of the microorganisms, for instance with oxygen, respectively for the removal of CO². This can also occur by threading gas perfuseable oxygenation hollow fibers taken from blood oxygenator production into the hollow pathway system. The media outflow is then secured via the third independent hollow pathway system. Alternatively the first and third hollow pathway system can be operated in counter current flow, whereby a cell perfusion is achieved through pressure gradients between the two systems. Afore mentioned channel like hollow pathway systems infuse the porous body of the described module. The dimension of the pores of the porous body of the module is selected in such a way that the pores exceed the size of a cultivated cell. The pores of the porous body therefore exhibit a diameter of preferably 10-1000 micrometer. Importantly, these pores communicate with each other via pore wall openings to facilitate an optimal in- and outflow of media across a multitude pores, whereby the pores are connected through openings of preferably 5-500 micrometer in size. This arrangement guarantees that the inflowing media can reach every part of the porous body via the independent hollow pathway systems, and like wise the outflowing media can be disposed of, via the pores and their connections to the channels of the hollow pathway system, from every part of the porous body. Therewith a media perfusion, flushing of cells, migration of cells as well as substrate exchange is possible through the pores. Therefore the porous body can also be referred to as an open porous foam-/sponge like structure. This bioreactor describes a device that facilitates the organ like reorganization of biological cells, especially in co-culture of parenchymal and non-parenchymal cells of an orgen.

The porous body that is arranged inside the casing can exhibit any geometrical shape. It is important that the porous body has a volume that is able to hold enough cells, respectively microorganisms, for various different applications. Therefore, the porous body exhibits a volume of preferably 0.5 ml-10 liters.

The geometrical shape is not determined. Preferred is a block form because it permits easy infusion of one hollow pathway systems from one side to the other and another hollow pathway system from an additional side. Preferred are cuboids or other rectangular hollow block forms.

Only modules exceeding three hollow pathway systems require a more complex outer form.

The porous body in block form can be generated from one piece, or the porous body is constructed form networks of several overlaying, disc/slide like, individual layers that are retained by the container.

In regards to afore mentioned second alternative, the disc/slide like arrangement, it is advantageous if at least one plane of the disc/slide like individual layers are infused with channel like ridges. These channel like ridges are arranged on the surfaces and shaped in such a way that they, in connection with the very next individual layer, form a channel like hollow pathway system. Therefore, the ridges are for instance shaped like a semi-channel so that, via interconnection with the next following individual layer, a complete channel is formed. The advantage of this arrangement is that it is technically very easy to equip the individual discs/slides with ridges. Preferably, the individual discs/slides can also be constructed in such a way that, viewed from the front wall, they exhibit the second channel like hollow pathway system in form of infused channels.

Consequently, the construction of these individual layers and their connections create a porous body with two independent hollow pathway systems. One hollow pathway system is created by the ridges in the individual layers, whereas the second hollow pathway system is created by the channel like hollow pathways already infused into the individual disc/slides. Drilling ridges into the remaining plane of the discs/slides can form a third hollow pathway system.

The porous body, as afore described, is arranged inside a casing. The configuration of a watertight-/germ tight container and open porous body is arranged in such a way that the channel-like hollow pathways of a system meet in at least one inflow and outflow heads. These inflow and outflow devices are configured in such a way that they pass through the container ensuring the supply and waste removal of the hollow body, arranged inside the container, from the outside. For this purpose two different designs are possible. One is that the inflow and outflow devices are part of the container itself and the arrangement of the body inside the container creates the connections. The other is to connect the inflow and outflow devices with the porous body, in which case the arrangement is enclosed by the sterile and water tight container.

The container can be in form of a solid casing or a foil. A container is the preferred application whereby the use of an injection-molding casing is advantageous. All known, state of the art materials, for example from polycarbonate, are possible for the injection-molding casing. It is advantageous if the container and the connections are constructed from bio-absorbable/bio-degradable material to potentiate the use of the module as medical implant.

Any known state of the art material can be used for the porous body that exhibits afore defined dimensions in regards to the pores and their connections, which leads to an open porous foam-/sponge like structure. As afore mentioned in connection with the container a biodegradable material can be used here as well.

Preferably, the material consists of sintered ceramic powder, especially the use of hydroxyapatite. Hydroxyapatite belongs to the group of calcium phosphates, which include ceramic materials with varying parts of calcium and phosphate. Hydroxyapatite is a compound that occurs in nature but can also be manufactured synthetically. The clinical use of hydroxyapatite as bone replacement material is an already know state of the art application. The motivation for the clinical use of hydroxyapatite is to apply a compound with a similar chemical composition as the mineral part of bone marrow. Hydroxyapatite exists in 60-70% as a natural component in the mineral part of the bone marrow. Hydroxyapatite powder can be generated via precipitation method from a watery solution, for instance by adding ammonium phosphate in a calcium nitrate solution and basic pH. A sintering process at 1000 to 2000 degrees Celsius will result in compounding the powder particles (Wintermantel et al.: Biokompatibler Werkstoff und Bauweise: Implantate fur Medizin und Umwelt, Berlin Springer 1998: 256-257). Wintermantel describes the manufacturing of a porous solid body from hydroxyapatite, for example open porous foam like structures, where hydroxyapatite powder is mixed with organic additives and then cauterized under high temperatures.

A further module that has also been described and simultaneously submitted with the description at hand, by the same inventors, titled “Bioreactor for cell self-assembly in form of an organ copy; procedures for the production and the application of cell culture, differentiation, maintenance, proliferation and/or use of cells.” (German patent application #103 26 746.8 of 13 Jun. 2003, J. Gerlach). In this case the bioreactor consists of a container that holds a open porous body whose pores also communicate with each other. In addition, the body contains at least two independent, branching out hollow pathway systems that cross and/or overlay each other and infuse the body. These hollow pathway systems depict natural organ copies, e.g. arteries and veins. Cells also settle inside the open pores of the body and are immobilized.

Therewith a bioreactor in form of an organ copy is made available. The hollow structures of the bioreactor allow for the maintenance of a larger cell mass with high density, whereby the fluid exchange to and from the cells via blood plasma or media occurs decentralized and avoiding large substrate gradients. The hollow structures include copies of arteries, veins, as well as other organ typical vessels for example liver portal veins of the liver, liver biliary tract canaliculi, and the Hering Channels with the liver stem cells.

Essential with this bioreactor is that its immunological inactive porous body exhibits open pores that communicate with each other. The pores exhibit a size that is larger then the size of the cells of the respective organ. Therefore the pores have a diameter of preferably 10-1000 micrometer and they are connected through pore wall openings. These openings, preferably formed channel- like, are preferably 5-500 micrometer in size. Through this arrangement the communication between the pores via the pore wall openings and with the hollow structures of the organ copy is secured. Via the pores a media perfusion, inflow of cells, cell migration as well as substrate exchange is made possible. Afore described structure of the porous body can also be referred to as an open porous foam-/sponge like structure. This bioreactor describes a device that facilitates organ typical reorganization of biological cells.

Importantly, the bioreactor is constructed from an immunological inactive, perfuseable open porous foam-/sponge like structure, whereby cells are settled inside the hollow spaces, and the pores of the foam-/sponge like structure. Via the pores media perfusion, inflowing of cells, cell migration as well as substrate exchange is made possible. Therewith, afore mentioned bioreactor is significantly improved with respect to known, state of the art, bioreactors in regards to mimicking substrate exchange structures, performances, and characteristics/attributes of natural organs.

This bioreactor describes a device that facilitates organ typical reorganization of biological cells. It is characteristic for this bioreactor that the specific hollow structures for the cell maintenance are arranged the same way as they occur in the natural organ.

All known state of the art materials, that produce open porous foam-/sponge like structures according to the invention, are well suited. Suitable are for instance ceramics, e.g. hydroxyapatite. Hydroxyapatite exists in form of a powder and, with additives and pore forming materials, can be frothed to foam-/sponge like structures and then sintered.

This bioreactor is preferably located in a sterile and water tight container. Suitable are foiled or accordingly dimensioned containers. In this case connections are provided, which are in connection with at least one hollow structure of the organ cast to guarantee the appropriate supply and waste removal in the bioreactor. In reference to the design of the connections, naturally several in-and outflow devices of the organ, inside the container, can be combined to one in-and outflow device.

In addition, it is advantageous with this bioreactor that the container and the connections can be generated from bio-absorbable, respectively biodegradable material which potentiates the use of the bioreactor as implant.

Afore described three registrations are, in their entirety, included in the registration at hand in regards to their disclosure content, design of the module/bioreactor, since such bioreactors can also be applied as bioreactor in the invention at hand.

Other bioreactors are already known from WO 00/75275 (Mac Donald, USA) and EP 1 185 612 (Mac Donald, USA).

Above described modules are generally suited for cell culture, proliferation and differentiation of cells, whereby the cells are encased in the respective containers of the modules and supplied through hollow pathway systems. Therewith, besides cell production, also the synthetic performances of the enclosed cells can be utilized, because the cell products can be led away from the reactor. However, the disadvantage of these bioreactors is, that they are not able to facilitate complex systems of cells in a circulation requiring the communication of several organs, or the migration between several organs. An example is the preservation of early stem cells in the bone marrow, maturation, or differentiation of immune cells at several further locations in the body. Hereunto the biological interactions in the organism with several independent organs within the blood/plasma circulation are much too complex. Particularly in the biological systems of the human body, the differentiating cells run through spatially varying stations that have to be passed through in a chronologically defined rhythm. During this process rest- and activity phases occur in different locations in the organisms in regards to cell differentiation. In addition, growth- and differentiation factors synthesized by various organ systems interact with each other via the circulatory system.

The invention at hand creates a hybrid circulatory system that implements such an interactive organ circuit structure.

This task is solved via the hybrid circulatory system according to claim 1 as well the application according to claim 37. Advantageous, advanced developments of the hybrid circulatory system are described in depending claims.

As per the invention, bioreactors are interconnected in a circulatory system, whereby a revolving media circuit ensures substrate exchange between at least two bioreactors. The substrate exchange can include mediators, soluble receptors, effectors, antibodies, and metabolic products like differentiation factors, growth factors, hormones, and such.

The substrate exchange can be controlled via the molecular cut off of the membranes used. This exchange can also include cell transfer. The cell exchange can also be controlled via the pore size of the membranes used

This invention permits cells to circulate between individual bioreactors. Thereby, for example, bone marrow cells can pass through the individual developmental stages as they occur in the human body. This means, differentiating bone marrow stem cells will first proliferate in a bioreactor providing a cell environment similar to bone marrow, from which they will be transported to a bioreactor with an environment corresponding to that of spleen tissue, or followed by a bioreactor that resembles the thymus and/or the liver. Then, the differentiating bone marrow stem cells are transported (or can actively migrate) into a bioreactor resembling the lymph nodes. It is also possible to, intermittently, set up small bioreactors with a cell specific environment resembling lymph nodes through which the cells have to pass.

The cell specific environment is generated in such a way that the differentiating cells are cultivated in co-culture with supporting cells of the respective organ like stroma cells, endothelial cells, and/or connective tissue cells. This can occur either inside the same compartment, via a semi permeable membrane (or a hollow fiber membrane structure) separate from the differentiating cells. In later case, the two compartments exchange mediators and effectors relevant for the differentiating cells that are generated by the cell specific environment.

Similarly, bioreactors with lymph node-like cell structures (or other organ typical bioreactors) can be connected with, for example, the circulatory system via a semi permeable membrane to restrict uptake into the circulatory system to certain mediators or effecters instead of cells.

The bioreactors cannot only be arranged in a row, but in copying the natural system, it is also possible to parallel arrange individual bioreactors into the circulatory system.

Alternatively it is possible to only circulate metabolic products of individual bioreactors in the circulatory system rather then circulating cells from one bioreactor to another. In this case it is possible to cultivate a particular cell in a stationary bioreactor, which will be supplied with mediators and effectors, necessary for their growth and proliferation, through other bioreactors that are connected to the circulatory system via a semi permeable membrane.

Thus it is also possible, for instance, to proliferate a stem cell culture and therewith produce stem cells in an indirect exchange with animal feeder cells. Furthermore, a human- to human stem cell/feeder cell structure can therefore be enabled. These techniques may be called compartmentalized co-culture.

Otherwise it is possible to generate certain mediators, effecters and such, and subsequently isolate them from the circulatory system. This is particularly advantageous when the respective mediators and effecters are not yet known, however under the given conditions can be generated as they occur in the biological body.

Thus it is possible to create a complete cycle of the maturation of, for instance, blood cells, the differentiation of immune cells, or the maintenance of proliferating stem cells inside a bioreactor. Should the circulatory system be set up to generate antigens, it is possible to produce immune cells that respond to antigens, which facilitates the production of vaccines.

Likewise it is possible to produce viruses, viral components or products that are necessary for the development of vaccines, which, in this context, are considered metabolic products of the cultivated cells.

Based on the complex interactions of organ systems in a human organism, the hybrid circulatory system permits the preservation of the early stem cells and their selective proliferation while conserving the early stem cell pool.

The invention permits the simulation of specific biological processes, for instance the growth of stem cells, stem cell differentiation by mediators produced in distant organ systems, cellular migration across lymphatic structures (spleen, lymph nodes), physiological migratory paths of the cells with ease and activity across several tissue stations, migration across tissue of different germ layers, as well as concluding proliferation and differentiation to immune cells or maturation to blood cells.

The circular media transfer of the circulatory systems can serve for the transfer of cells or the transfer of cellular signals, respectively chemical mediators or signals between the bioreactors and tissue structures. An analogous transfer can also occur within one reactor that contains two different compartments, for example one compartment for the culture of cell lines and another compartment for the co-culture for an organ specific environment, simulating the in vivo macro environment of individual cell lines. Additionally, a selective contact of individual cells in a bioreactor, with defined molecules of determined size, can be achieved via the technical, in any pore size set, exclusion barrier of individual molecules into the bioreactor.

Following, a few examples of hybrid circulatory systems are explained:

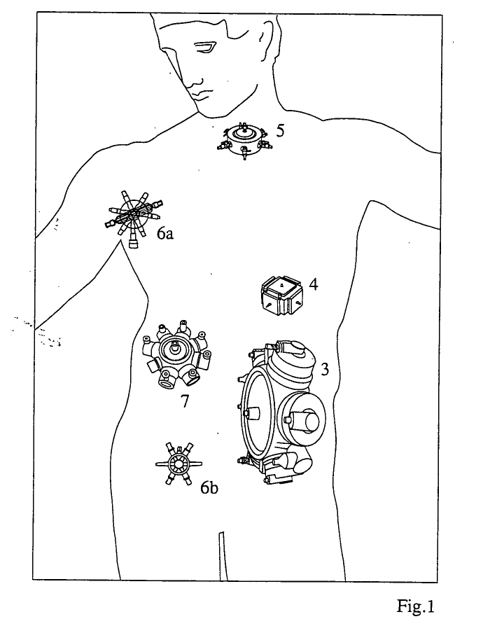

FIG. 1 describes various bioreactor systems analogous to human organs

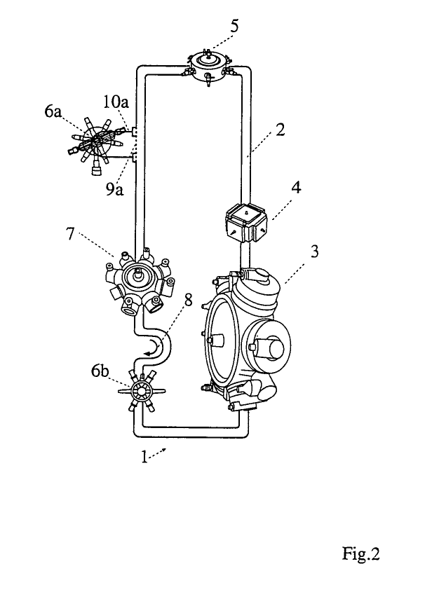

FIG. 2 describes a hybrid circulatory system

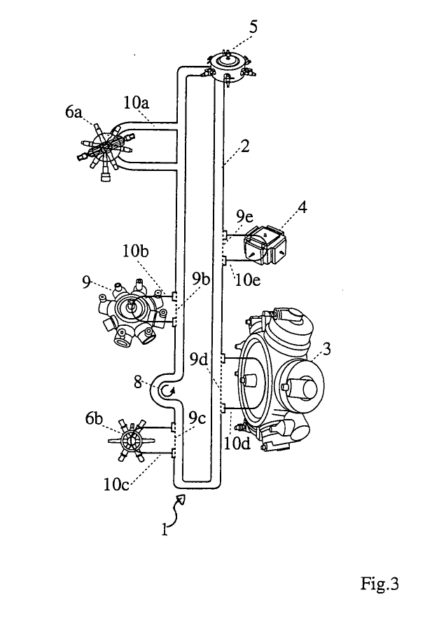

FIG. 3 describes another hybrid circulatory system

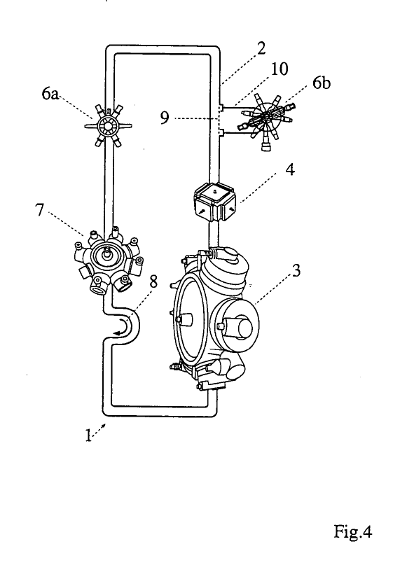

FIG. 4 describes another hybrid circulatory system

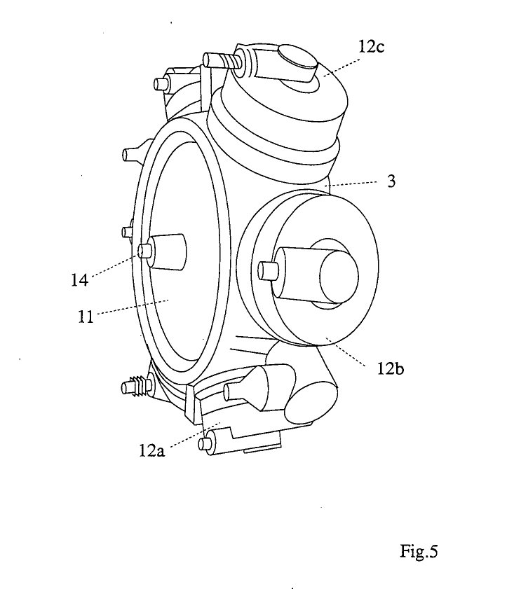

FIG. 5-10 describes a schematic drawing of a bioreactor, built for and used in the hybrid circulatory systems in FIG. 2 and 3

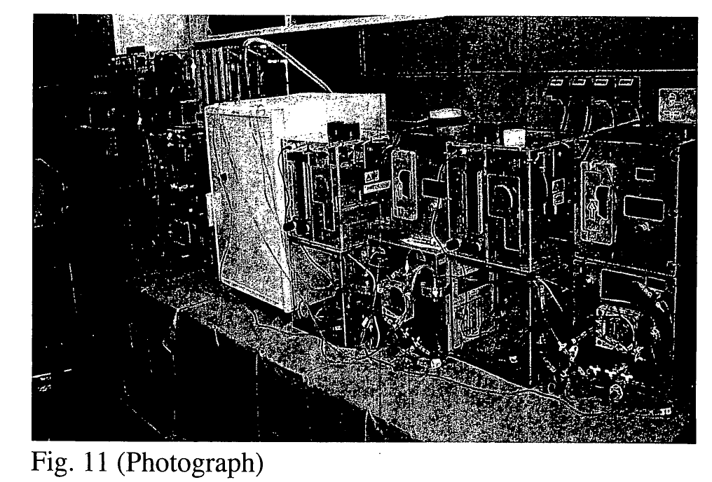

FIG. 11 shows a photograph of an experiment with a hybrid circulatory system. In order to explain the features, a schematic drawing follows with numbers.

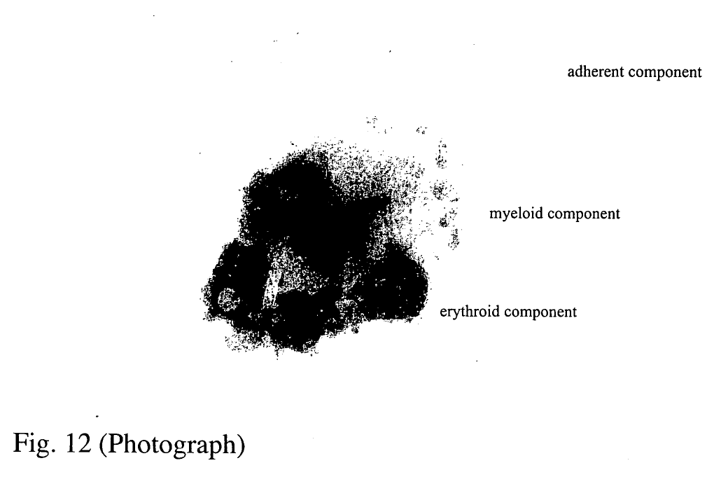

FIG. 12 describes the photograph and a schematic drawing of a colony of blood cells from the hybrid circulatory system

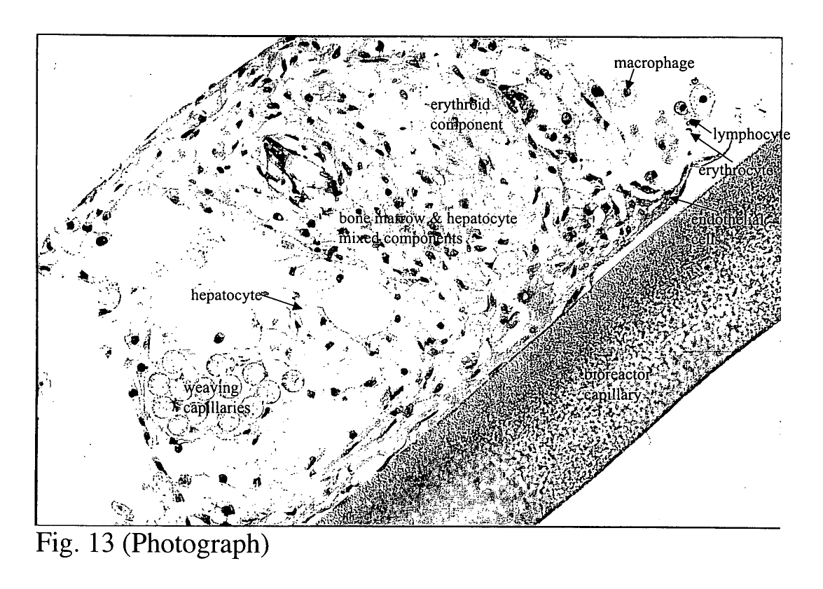

FIG. 13 shows a photograph and a schematic drawing describing the blood cell differentiation in a bioreactor, under co-culture of bone marrow immune cells and liver cells in the circulatory system

In FIG. 1 describes 3 a bioreactor in which bone marrow cells are cultivated. The reactors 4, 5, 6a, 7, and 6b describe bioreactors cultivating spleen cells (reactor 4), thymus cells (reactor 5), liver cells (reactor 7), and lymph cells (reactor 6a and 6b).

These reactors describe the essential elements o a circulatory system in which bone marrow cells can be cultivated, proliferated, and differentiated.

FIG. 2 describes such a fully developed system, whereby the bioreactors 3, 4, 5, 7 and 6b are interconnected via a ring line 2, each being infused through these ring lines via inflow devices. Additionally, a reactor 6a is connected to the circulatory system via another circuitry 10a and a semi permeable hollow fiber membrane 9a. The reactor is infused through the circuitry 10a via inflow devices so that differentiation factors generated in the lymph node cells in reactor 6a, mediators, growth factors and such can be released from the circulatory system 10a into the ring line 2via the semi permeable membrane. The ring line is constantly flushed because the media flowing within is continuously recycled through a pump 8. In this circulatory system 1 of FIG. 2, bone marrow immune cells from the reactor 3, in which cells are cultivated in a bone marrow specific environment, can migrate through the reactors 4, 5, 7, and 6a, thereby passing through independent organ specific environments of each bioreactor, depending on the cells that are cultivated/co-cultured in the respective reactors. This permits the bone marrow immune cells to pass though all gestation processes in the right order and chronology, and as a result differentiate into complete immune cells. It is possible to connect additional bioreactors to the ring line 2 via the semi permeable membrane to cause the release of mediators or effecters into the ring line 2 through a semi permeable membrane.

The interaction of the bone marrow cells inside the bioreactor 3 with the cells/mediators of other bioreactors also facilitates the preservation of the early bone marrow stem cells ensuring the long-term conservation/preservation of the entire system.

FIG. 3 describes an alternative to FIG. 2, in which the ring line 2 only directly flows through the reactor 5 and in a side branch flows through reactor 6a who exhibits a lymph node specific environment. The reactors 7, 6b, 3, and 4 are, via semi permeable membranes 9b, 9c, 9e, 9d, and their own ring lines 10b, 10c, 10d, 10e for substrate exchange, connected with ring line 2. The semi permeable membranes 9b through 9e are arranged in such a way that the pores release mediators/effecters, which are generated in the reactors 7, 6b, 3, 4, 5, into the media that flows in the ring line 2. The bone marrow reactor 3, for example, contains bone marrow stem cells that are supplied with all mediators and effecters from the individual organ specific reactors through the semi permeable membrane 9d and the ring line 10d.

Alternatively, this circulatory system from FIG. 3 can also be arranged in such a way that, as in FIG. 2, the reactor 3 can be directly infused so that the differentiating bone marrow immune cells can circulate in ring line 2. The individual levels of differentiation are thereby induced that the appropriate effecters from the other reactors are flushed into the ring line 2 via the semi permeable membrane, as afore described. Such switching of integration of semi permeable can be realized for instance by using two three-way valves.

FIG. 4 describes a further circulatory system 1, in which the reactors 3, 4, and 7 are arranged in a ring line 2 as shown in FIG. 2. The reactor 6a is also directly arranged inside the ring line 2 and is infused with media from the ring line 2 through inflow devices. In between the reactor 4 and reactor 6a a reactor 6b is located that simulates a lymph node, which on its part is connected, via a branched ring line 10, with the ring line 2 for the exchange of mediators and metabolic exchange products/nutrients.

FIG. 5 describes a schematic drawing of a photograph of a reactor 3, in which the main body 12 and the inflow devices 12a, 12b, and 12c for the maintenance and waste management of the cell culture located inside the main body 12. Reference mark 14 marks an inflow through which the inside of the reactor 3, respectively its cell compartment, can be directly infused.

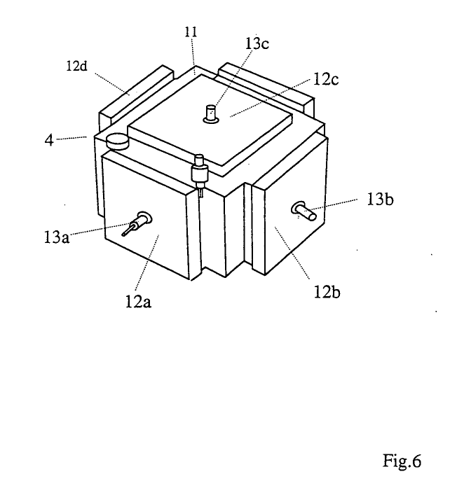

FIG. 6 describes a schematic drawing of a photograph of a reactor 6 whereby the appropriate inflow devices 12a, 12b, and 12d are recognizable, and through which hollow fiber membranes inside the main body 11 of the reactor 4 can be supplied with nutrients, mediators, growth factors and such, and at the same time metabolic exchange products can be removed. Inflow device 12c with connection 13c creates the direct connection to the cell compartment into which cells can migrate in or out.

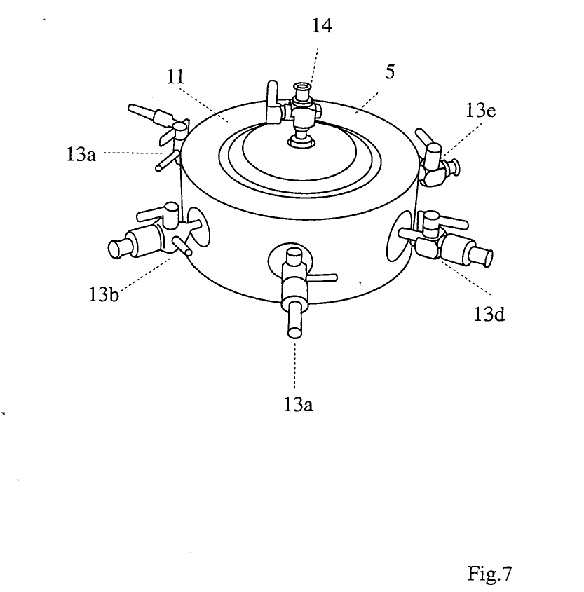

FIG. 7 describes the bioreactor 5 marking 13a through 13e as inflow connections with which the main body 11 inside can be supplied with substances necessary for the metabolism, culture, and proliferation of thymus cells, and at the same time metabolic exchange products can be removed. The metabolic exchange products can then be flush into the ring line 2.

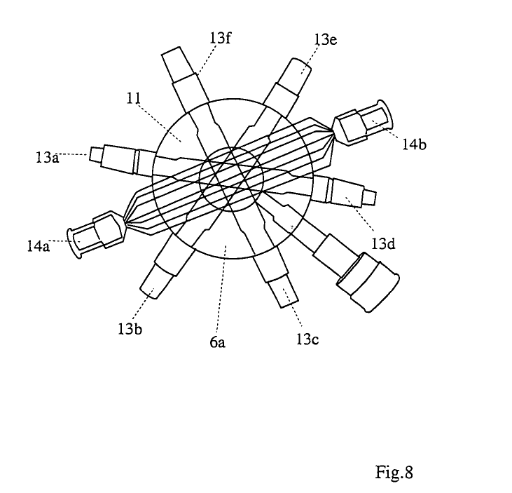

FIG. 8 describes a reactor 6a, whereby the same reference marks mark similar element s as in FIG. 7.

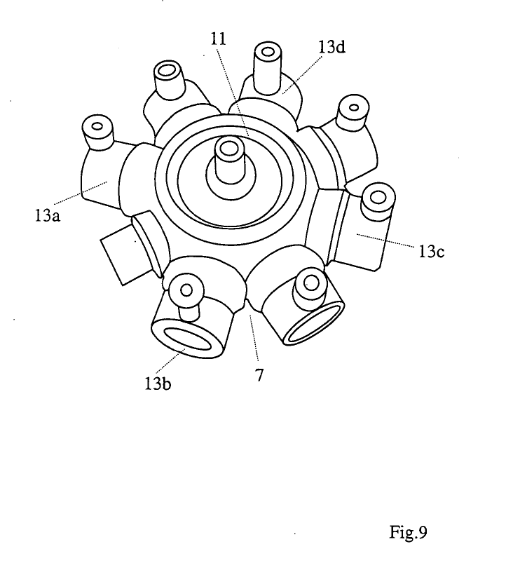

FIG. 9 describes a further reactor 7, which is arranged in a similar way as the reactor depicted in FIG. 5. This reactor serves for the cultivation of liver cells and to create a liver specific environment for the differentiating cells migrating in and out of the reactor. Alternatively, this reactor serves for the generation of effecters or mediators through the liver cells that are either needed by other cells for their further development or are removed and utilized as end product. Likewise, in the liver reactor 7, liver cells can be extracted and differentiated or liver stem cells can be proliferated for later therapeutic or other use.

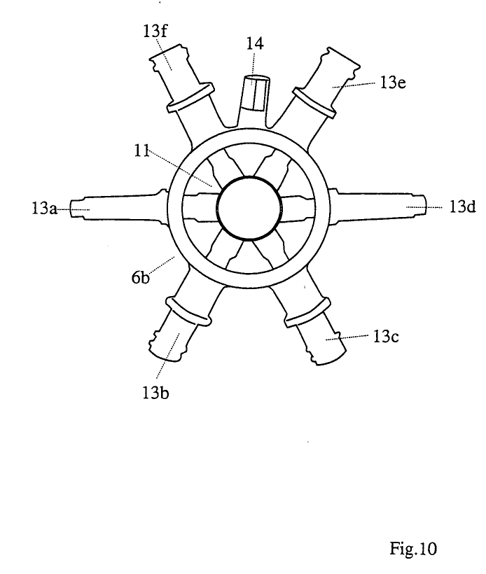

FIG. 10 describes a further reactor 6b, which is used to generate lymph node specific cell cultures.

In the invention and the circulatory system, it is ideal that each reactor contains, proliferates, and/or differentiates the necessary organ specific cells.

Overall, the circulatory system is able to imitate not only the circulatory system of the body but also the entire system of the blood circuit and organs.

FIG. 11 describes a photo and a subsequent drawing of a circulatory system. The arrangement in FIG. 11 shows that at least three units 20a, 20b, 20c are interconnected, whereby the basic structure of each unit 20a through 20c is identical. Each one of the units 20a through 20c contains a unit 21a through 21c with one afore described reactor. Reference mark 21a marks a reactor according to FIG. 9 for bone marrow, whereas the reference marks 21b and 21c mark a reactor according to FIG. 10 for liver cells, respectively liver cells and bone marrow cells. Units 20a through 20c also exhibit a fresh media pump 22a through 22c as well as a circulatory pump 23a through 23c. The circulatory pump 23a through 23c passes the media between the reactors in the circulatory system.

As fourth component of each unit 20a through 20c, unit 24a, 24b, and 24c is added with which the temperature of all system components is controlled via warm air.

A refrigerator maintaining a temperature of 4 degrees Celsius is made available for all units 20a through 20c in which for instance the fresh media supply is stored. A medium circulation is arranged between the individual units 20a through 20c so that the bioreactors 21a through 21c, contained in units 20a through 20c, can exchange substrates or cells.

The three-way valves in the circulatory system can be positioned either to infuse the individual cell systems directly via the cell compartment, or via semi permeable membrane.

FIG. 12 describes a microphotograph of an individual bone marrow cell, and a subsequent schematic drawing that was extracted from a bioreactor 3 as afore described, which generated a colony of various blood cells

FIG. 13 describes a microphotograph of a section through a co-culture in a reactor 7 and a subsequent schematic drawing. In this reactor bone marrow immune cells were cultivated in co-culture with liver cells. It is obvious from FIG. 13 that, in co-culture with the hepatocytes, the bone marrow stem cells were differentiated into lymphocytes as well as erythrocytes. It is obvious that the co-culture with the hepatocytes created the appropriate organ specific environment to facilitate the differentiation of bone marrow cells.

1. Hybrid organ circulatory system (1) with at least two bioreactors (3 through 7), that are arranged in such a way that living cells can be cultivated, differentiated and/or proliferated inside them, whereby at least the two bioreactors (3 through 7) are connected with each other through a circular-shaped media line (2) to allow for cell and/or substrate exchange between the bioreactors (3 through 7).

2. Hybrid organ circulatory system (1), according to afore mentioned claims, is thereby characterized that the cell compartment of at least one bioreactor (3 through 7) is directly perfuseable via the circular-shaped media line (2).

3. Hybrid organ circulatory system (1), according to one afore mentioned claims, is thereby characterized that at least one of the bioreactors (6a) is perfused by a second media circuit line. The lumina of the second media line (10a) and the circular-shaped media line (2) are in substrate exchange via a semi permeable membrane (9a), whereby the pore size, or the molecular weight cut-off, of this membrane is adjustable to allow passage for molecules or cells up to a certain size.

4. Hybrid organ circulatory system (1), according to one afore mentioned claim, is thereby characterized that at least one of the bioreactors (3 through 7) is divided into mass exchange compartments by means of at least one membrane or sieve like structure.

5. Hybrid organ circulatory system (1), according to one of the two afore mentioned claims, is thereby characterized that the membrane is semi permeable, respectively the sieve like structure is only permeable to substances or cells with a diameter smaller then a predetermined pore diameter, or molecular weight cut-off.

6. Hybrid organ circulatory system (1), according to afore mentioned claims, is thereby characterized that the semi permeable membrane (9a) is permeable for the media, biological cells and/or substances.

7. Hybrid organ circulatory system (1), according to afore mentioned claims, is thereby characterized that the semi permeable membrane is permeable for media and substances but not for biological cells.

8. Hybrid organ circulatory system (1), according to one of the two afore mentioned claims, is thereby characterized that the semi permeable membrane (9a) is permeable for nutritive factors, metabolic factors, differentiating factors, signal factors, cytokines, mediators, hormones, antibodies and such substances.

9. Hybrid organ circulatory system (1), according to one of afore mentioned claims, is thereby characterized that at least one of the bioreactors (3 through 7) exhibits a module for the culture and utilization of metabolic activity, production and/or maintenance of microorganisms, especially for cells consisting of an outer casing, at least two independent membrane systems, whereby at least one independent membrane system is arranged as a hollow fiber membrane system arranged inside the module.
The hollow fiber membranes form a tightly packed spatial network, and the microorganisms that are located in the spaces of the network and/or adhere to the hollow fiber membranes (3), whereby the network consists of intersecting and/or overlaying hollow fiber membranes and is constructed in such a way that the microorganisms have almost identical conditions of substrate supply -and removal from every point inside the module (1).

10. Hybrid organ circulatory system according to claim 9, is thereby characterized that the tightly packed network in the interior of at least one of the bioreactors is constructed from three independent hollow fiber membrane systems.

11. Hybrid organ circulatory system according claims 9 or 10, is thereby characterized that in addition, an exchangeable flat membrane or capillary membrane or sieves mounted on the outer casing and has access to the cell compartment.

12. Hybrid organ circulatory system (1), according to claim 9 through 11, is thereby characterized that in addition the tightly packed network exhibits another fluid impermeable independent capillary system.

13. Hybrid organ circulatory system (1), according to claim 9 through 12, is thereby characterized that the outer casing is generated from a cast, whereby entry into the lumen of the capillaries or hollow fiber membranes is made possible.

14. Hybrid organ circulatory system (1), according to claim 9 through 13, is thereby characterized that for the in- and outlet into the lumen of the capillaries or hollow fiber membranes corresponding in- and/or outlet heads (6, 13, 14, 15) are provided that are communicating with the respective capillary system.

15. Hybrid organ circulatory system (1), according to claim 9 through 14, is thereby characterized that several entries are provided in the outer casing of the module that lead inside to flush microorganisms into or out of the module, and/or conduct pressure-temperature-, fluorescent light-, and /or pH-measurements, and/or the application of movements/flow/pressure to support cell harvest, and/or are thereby identified that cell migration is provided by utilizing at least two entries into the cell compartment along the perfusion line, out of and into the cell compartment.

16. Hybrid organ circulatory system (1), according to claim 15, is thereby characterized that that the inlets continue into the module as perforated tubes which allow for an even distribution of the microorganisms in the cell compartment.

17. Hybrid organ circulatory system (1), according to on of the afore mentioned claims, is thereby characterized that at least one of the bioreactors (3 through 7) exhibits a module for the culture and utilization of metabolic activity, proliferation and/or the maintenance of microorganisms, especially for cells consisting of an open porous body, whose pores communicate with each other, that is arranged inside a water- and germ tight container. This porous body should be infused with at least one channel-like hollow pathway system whose individual hollow pathways intersect and/or overlay each other inside the body.

18. Hybrid organ circulatory system (1), according to claim 17 is thereby characterized that it exhibits at least two independent channel-like hollow pathway systems.

19. Hybrid organ circulatory system (1), according to claim 18, is thereby characterized that a channel-like hollow pathway system consists of at least one plane arranged with parallel running individual channels.

20. Hybrid organ circulatory system (1), according to claim 19, is thereby characterized that a hollow pathway system consists of several planes layered on top of each other that consist of parallel running individual channels.

21. Hybrid organ circulatory system (1), according to one of the claims 18 through 20, is thereby characterized that three independent hollow pathway systems are available.

22. Hybrid organ circulatory system (1), according to one of the claims 18 through 21, is thereby characterized that four independent hollow pathway systems are available.

23. Hybrid organ circulatory system (1), according to at least one of the claims 17 through 22, is thereby characterized that the diameter of one individual channel of the channel-like hollow pathway system is 0.1-3 mm.

24. Hybrid organ circulatory system (1), according to at least one of the claims 17 through 23, is thereby characterized that the spacing, of the parallel running channels of a hollow pathway system arranged in one individual plane and/or between planes, is 0.5-5 mm.

25. Hybrid organ circulatory system (1), according to at least one of the claims 17 through 24, is thereby characterized that the open pores of the body have a diameter of 10-1000 micrometer.

26. Hybrid organ circulatory system (1), according to at least one of the claims 17 through 25, is thereby characterized that the open pores are connected through openings of 10-500 micrometer in size.

27. Hybrid organ circulatory system (1), according to at least one of the claims 17 through 26, is thereby characterized that the body is a network of several, each other overlaying, disc/slide like individual layers, which are retained by the container.

28. Hybrid organ circulatory system (1), according to at least one of the claims 17 through 27, is thereby characterized that at least one surface of the disc/slide like individual layers is infused with channel like ridges, which are arranged and dimensioned in such a way that, in connection with the following individual layer, a channel-like hollow pathway system is formed.

29. Hybrid organ circulatory system (1), according to at least one of the claims 27 through 28, is thereby characterized that the front wall of the disc/slide like individual layers are infused with a channel-like hollow pathway system.

30. Hybrid organ circulatory system (1), according to claim 29, is thereby characterized that the disc/slide like individual layers are infused with hollow pathways from one surface to the next.

31. Hybrid organ circulatory system (1), according one of the claims 17 through 30, is thereby characterized that the channel-like hollow pathways of a system meet in at least one inlet and outlet.

32. Hybrid organ circulatory system (1), according to claim 31, is thereby characterized that the inlet and outlet is connected with the porous body.

33. Hybrid organ circulatory system (1), according to claim 31, is thereby characterized that the inlet and outlet is part of the container.

34. Hybrid organ circulatory system (1), according to claims 17 through 33, is thereby characterized that the walls of the open porous material consists of a sintered ceramic powder.

35. Hybrid organ circulatory system (1), according to one of the afore mentioned claims, is thereby characterized that at least one of the bioreactors is in form of a perfuseable organ copy that consists of organ-specific hollow pathway structures and an immunological inactive open porous body whose open pores communicate with each other.

36. Hybrid organ circulatory system (1), according to claim 35, is thereby characterized that the pores of the bioreactor have a diameter of 10-1000 micrometer.

37. Hybrid organ circulatory system (1), according to claims 35 or 36, is thereby characterized that the pore wall openings of the open porous structure have a diameter of 5-500 micrometer.

38. Hybrid organ circulatory system (1), according to claims 35 through 37, is thereby characterized that the organ copy is arranged inside a water- and germ tight container and that the outer casing is equipped with connectors that are in contact with at least one hollow structure of the organ copy.

39. Hybrid organ circulatory system (1), according to at least one of the claims 35 through 38, is thereby characterized that the container and the connections consist of biodegradable material.

40. Hybrid organ circulatory system (1), according to at least one of the claims 35 through 37, is thereby characterized that the porous body consists of biodegradable material.

41. Hybrid organ circulatory system (1), according to one of the claims 35 through 37, is thereby characterized that the pore walls of the open porous body consists of a sintered ceramic powder.

42. Hybrid organ circulatory system (1), according to one of the claims 35 through 41, is thereby characterized that it is a copy of the liver, bone marrow, lymph nodes, thymus, spleen, kidney, pancreas, islets, mucosa, thyroid, adrenal glands, bone, gonads, uterus, placenta, ovaries, testis, blood vessels, heart, lungs, muscle, intestinal wall, bladder, heart muscle, and/or additional mammalian organs.

43. Hybrid organ circulatory system (1), according to one afore mentioned claims, is thereby characterized that inside each of at least two bioreactors (3 through 7) first cells of a predetermined organ, respectively predetermined type are settled.

44. Hybrid organ circulatory system (1), according to afore mentioned claim, is thereby characterized that at least one of the bioreactors contains embryonal stem cells, fetal stem cells, primary adult stem cells, cell lines, immortalized cells, gene technologically modified cells, feeder cells, and/or adult mammalian cells.

45. Hybrid organ circulatory system (1), according to claim 13, is thereby characterized that at least one of the bioreactors (3) contains precursor cells of bone marrow cells or cells that derived from such precursor cells through maturation, respectively differentiation.

46. Hybrid organ circulatory system (1), according to afore mentioned claims, is thereby characterized that the first cells in at least one of the bioreactors (3) are bone marrow stem cells prior to developing immune competence and/or blood cells, respectively immune cells during maturation, respectively differentiation.

47. Hybrid organ circulatory system (1), according to one of afore mentioned claims, is thereby characterized that the first cells in at least one of the bioreactors are kept in co-culture with additional cells of a different type.

48. Hybrid organ circulatory system (1), according to afore mentioned claims, is thereby characterized that the additional cells are non-parenchymal cells, or feeder cells.

49. Hybrid organ circulatory system (1), according to one afore mentioned claims, is thereby characterized that the additional cells create an organ typical organ environment for the first cells.

50. Hybrid organ circulatory system (1), according to afore mentioned claim, is thereby characterized that inside the bioreactor a biomatrix has been developed from a co-culture with non-parenchymal cells or stroma cells of a predetermined organ.

51. Hybrid organ circulatory system (1), according to one of the claims 47 through 50, is thereby characterized that the additional cells are releasing growth factors, differentiation factors, hormones, and/or other mediators.

52. Hybrid organ circulatory system (1), according one of the claims 47 through 51, is thereby characterized that the first cells and the additional cells are arranged in various compartments of a bioreactor.

53. Hybrid organ circulatory system (1), according to one of the claims 47 through 51, is thereby characterized that the first cells and the additional cells are arranged in various bioreactors.

54. Hybrid organ circulatory system (1), according to one of the two afore mentioned claims, is thereby characterized that the various compartments and/or the various bioreactors are connected in such a way that between the various compartments/bioreactors substances, like growth factors, hormones, differentiation factors and/or mediators, and/or first cells, and/or second cells can be interchanged.

55. Hybrid organ circulatory system (1), according to one of the claims 47 through 54, is thereby characterized that the first cells are, e.g. bone marrow stem cells and the additional cells are bone marrow stroma cells, vascular endothelial cells and/or cells of various germ layers; or in another example embryonic stem cells and the additional cells are feeder cells.

56. Hybrid organ circulatory system (1), according to one of afore mentioned claims, is thereby characterized that the bioreactors (3 through 7) exhibit an organ typical environment for each of the following organs: bone marrow, spleen, thymus, lymph nodes, uterus, placenta, ovaries, testis, and/or liver.

57. Hybrid organ circulatory system (1), according to afore mentioned claim, is thereby characterized that a bioreactor (6a, 6b) with a lymph node specific environment is located between each one or several of the bioreactors (3, 4, 5, 7).

58. Hybrid organ circulatory system (1), according to one of the two afore mentioned claims, is thereby characterized that in each such bioreactors differentiated cells of the respective organs are cultivated to generate an organ specific environment.

59. Hybrid organ circulatory system (1), according to one of the claims 56 through 58, is thereby characterized that inside the bioreactor, bone marrow precursor cells are cultivated in co-culture with bone marrow stroma cells in a bone marrow specific environment.

60. Hybrid organ circulatory system (1), according to one afore mentioned claims, is thereby characterized that substances, generated by cells, are transported in the media line (2) of the individual bioreactors (3 through 7) as bioreactor product from one bioreactor to another.

61. Hybrid organ circulatory system (1), according to one afore mentioned claims, is thereby characterized that at least on of the bioreactors (6a), whose products are transportable in the media line, is separated from the media line through a membrane or sieve like structure that is permeable only for the mediators that have to be transported.

62. Hybrid organ circulatory system (1), according to one afore mentioned claims, is thereby characterized that cultured, proliferating and/or differentiating cells are transportable in the media line from a bioreactor (3, 4, 5, 7) to the next bioreactor.

63. Hybrid organ circulatory system (1), according to afore mentioned claim, is thereby characterized that the interior of the bioreactor (3, 4, 5, 7), from which cells can be transported to other reactors, are perfused by the media line.

64. Hybrid organ circulatory system (1), according to one of the two afore mentioned claims, is thereby characterized that the cultured, proliferating and/or differentiating cells migrate within the circulatory system from bioreactor (3, 4, 5, 7) to bioreactor. Thereby they cycle through the natural stages of development in regards to the organ specific environment of the respective bioreactor in the appropriate order and timely course. The pore size of the sieves and/or membranes of the system define the maximum cell size of the migrating cells.

65. Hybrid organ circulatory system (1), according to one afore mentioned claims, is thereby characterized that the circulatory system contains antigens.

66. Hybrid organ circulatory system (1), according to afore mentioned claim, is thereby characterized that the antigens are contained in a media line (2) and/or in at least one of the bioreactors (3 through 7).

67. Utilization of a circulatory system (1), according to one afore mentioned claim for the production of substances like cellular metabolic products, known or unknown mediators, hormones, differentiating factors, signal molecules, growth factors, sensitization factors, cytokines, proteins, antibodies, vaccines, viruses and/or for the production of organ specific biomatrix substances.

68. Utilization of a circulatory system (1), according to one afore mentioned claim for development of a hybrid gland.

69. Utilization of a circulatory system (1), according to one afore mentioned claims for the generation of biological cells like stem cells, or differentiating cells, of a specific organ, blood cells, immune cells and/or embryonic cells.

70. Utilization of a circulatory system (1), according to one afore mentioned claims as hybrid gland for the production immune competent cells and vaccines, progenitor cells for organs, blood cells, such as blood platelets.

71. Utilization of a circulatory system (1), according to one afore mentioned claims as hybrid blood cell system (bone marrow) for the production of blood cells, especially blood platelets and erythrocytes.

72. Utilization of a circulatory system (1), according to one afore mentioned claims as hybrid stem cell system for the production of progenitor cells for organs, especially for the transplantation of repair cells.

73. Utilization of a circulatory system (1), according to one afore mentioned claim in cell based therapy, regenerative medicine, cell biology, vaccine development, expansion, proliferation, and differentiation of embryonic stem cells.
